(19) United States
(12) Patent Application Publication
(10) Pub. No.: US 2022/0213140 A1
(43) Pub. Date: Jul. 7, 2022

(54) METHOD FOR PURIFYING FC REGION-MODIFIED ANTIBODY

(71) Applicants: Chugai Seiyaku Kabushiki Kaisha, Tokyo (JP); ProteNova Co., Ltd., Kagawa (JP)

(72) Inventors: Tetsuya WAKABAYASHI, Shizuoka (JP); Yuichiro SHIMIZU, Singapore (SG); Masahiro FUKUNAGA, Tokyo (JP); Eiji MAJIMA, Kagawa (JP)

(21) Appl. No.: 17/602,196

(22) PCT Filed: Apr. 9, 2020
(86) PCT No.: PCT/JP2020/015904

§ 371 Date: Oct. 7, 2021

(30) Foreign Application Priority Data
Apr. 10, 2019 (JP) 2019-074738

(51) Int. Cl. C07K 1/22; C07K 16/28; B01D 15/38

(52) U.S. Cl. CPC C07K 1/22; B01D 15/3809; C07K 16/283

(57) ABSTRACT

The present inventors discovered affinity purification resins having sufficient binding affinity for Fc region variants with reduced binding to Protein A. Specifically, immunoglobulins containing an Fc region variant having reduced binding to Protein A could be purified using a Protein A-modified ligand containing a structure in which the amino acids of the C-domain have been substituted as an Fc ligand.

### TECHNICAL FIELD

The present invention relates to methods of purifying an antibody and to methods for purifying an Fc region-modified antibody comprising specific amino acid mutations.

### BACKGROUND ART

With the development of gene recombination technology, various protein preparations can now be supplied in stable amounts, and various therapeutic antibodies are being developed.

When an antibody is produced using mammalian cells as host by gene recombination technology, it is subjected to Protein A or Protein G affinity column chromatography by utilizing the property of Protein A or Protein G to bind to the Fc chain of IgG, after which, purification is carried out by various chromatographies. In particular, purification of an antibody by Protein A affinity column chromatography is the process most commonly used in the production of therapeutic antibody to recover the antibody from the culture medium.

For example, in JP-A (Kohyo) H05-504579 (PTL 1), an antibody-containing aqueous medium obtained from a mammalian cell culture was applied to Protein A or Protein G column chromatography to allow adsorption of the antibody onto the column, then the antibody was eluted with an acidic solution (citric acid with a concentration of about 0.1 M, pH 3.0-3.5), and the obtained acidic eluate was sequentially applied to ion exchange column chromatography and to size exclusion column chromatography for purification.

On the other hand, for the purpose of improving blood retention or in vivo kinetics, amino acid substitution techniques for regulating the isoelectric point (pI) of an antibody, specifically, techniques for regulating the pI of an antibody by modifying amino acid residues exposed on the surface of an antibody, are known (WO 07/114319 (PTL 2), WO 2017/104783 (PTL 3)). PTL 2 discloses that modification of amino acid residues of an antibody to regulate the pI is expected to improve plasma retention and half-life of the antibody, and that this leads to a reduction of the dose and the extension of the administration interval of the antibody as a medicament. Further, PTL 3 discloses that by introducing the amino acid substitutions Q311R and P343R into the CH2 region and CH3 region of an antibody to modify the antibody to increase the pI, antigen elimination from plasma can be enhanced when the antibody is administered in vivo.

Protein A used for antibody purification is a protein present in the cell wall of Staphylococcus aureus and binds to immunoglobulins, especially to the Fc region of IgG. In general, the Protein A protein derived from Staphylococcus has a repeated structure including five immunoglobulin-binding domains having homology to each other, called the E-domain, D-domain, A-domain, B-domain, and C-domain, and each binding domain can bind singly to an immunoglobulin. Along with natural Protein A, recombinant proteins consisting only of immunoglobulin-binding domain(s) with partially-modified amino acids are also used as affinity ligands for affinity chromatography. For example, Protein A columns with improved antibody purification efficiency have been developed by substituting any one or more originally present lysines at positions 4, 7, and 35 of a C-domain variant or Z-domain with an amino acid other than lysine, where the C-domain variant was prepared by substituting the glycine at position 29 of the amino acid sequence of the C-domain of Staphylococcus Protein A with alanine, and the Z-domain was prepared by substituting the glycine at position 29 of the amino acid sequence of the B-domain of Staphylococcus Protein A with alanine (PTLs 4 and 5).

### CITATION LIST

### Patent Literature

[PTL 1] Japanese Patent Application Kohyo Publication No. (JP-A) H05-504579 (unexamined Japanese national phase publication corresponding to a non-Japanese international publication)

[PTL 2] WO 2007/11431

[PTL 3] WO 2017/104783

[PTL 4] Japanese Patent Application Kokai Publication No. (JP-A) 2007-252368 (unexamined, published Japanese Patent Application)

[PTL 5] WO 2015/034000

### SUMMARY OF INVENTION

### Technical Problem

Protein A has been conventionally utilized as a ligand for antibody purification. However, evaluation of purification methods suitable for antibodies with amino acid modifications for modifying pI (hereinafter referred to as pI-modified antibodies) and issues in the purification process have not been investigated in detail so far. Therefore, for example, it was not known that there are antibodies that cannot be purified by commonly-used Protein A columns

The present inventors discovered that there are pI-modified antibodies that cannot be efficiently purified by the commonly-used Protein A columns An efficient purification method suitable for such antibodies is thus needed. In other words, it is an objective of the present invention to provide a highly efficient and economical purification method which enables production of an antibody on an industrial scale even when the antibody is a pI-modified antibody that cannot be efficiently purified by a common Protein A column.

Solution to Problem

As a result of diligent research to achieve the above objective, the present inventors discovered that the use of a resin comprising a specific modified Protein A ligand enables efficient purification of even pI-modified antibodies that cannot be efficiently purified with a commonly-used Protein A column.

More specifically, the present invention provides the following [1] to [20]:
[1] a method of purifying an IgG antibody comprising the amino acid residue substitutions Q311R and P343R from a composition containing the antibody, wherein the method comprises the steps of:
   (a) preparing an affinity column containing a carrier onto which a Protein A-modified ligand is immobilized, wherein the Protein A-modified ligand comprises: a modified immunoglobulin-binding domain comprising a modification for substitution of any one or more originally present lysine residues at positions 4, 7, and 35 of the C-domain variant of Staphylococcus Protein A of SEQ ID NO: 1 or Z-domain of Staphylococcus Protein A of SEQ ID NO: 2 with amino acid residues other than lysine; or a multimer of these modified immunoglobulin-binding domains;
   (b) loading the composition containing the IgG antibody onto the affinity column of step (a); and
   (c) eluting and recovering the IgG antibody from the affinity column of step (b);
[2] the method of [1], wherein the multimer of the modified immunoglobulin-binding domains is a dimer to decamer, and wherein arranged at the first or second from the N-terminal or C-terminal side in the multimer is an immunoglobulin-binding domain in which at least one of the originally present amino acid residues at positions 40, 43, 46, 53, 54, and 56 of the C-domain variant (SEQ ID NO: 1) or Z-domain (SEQ ID NO: 2) has been substituted with a lysine residue;
[3] the method of [1] or [2], wherein the substitution is a modification for substitution of any one or more originally present lysine residues at positions 4, 7, and 35 of the C-domain variant or Z-domain of Protein A with any one of amino acid residues selected from the group consisting of an alanine residue (A), a glutamine residue (Q), an asparagine residue (D), a valine residue (V), a serine residue (S), a threonine residue (T), a histidine residue (H), a tyrosine residue (Y), an arginine residue (R), a glutamic acid residue (E), a phenylalanine residue (F), a leucine residue (L), an isoleucine residue (I), and a proline residue (P);
[4] the method of any one of [1] to [3], wherein the modified immunoglobulin-binding domain is (i) a modified immunoglobulin-binding domain comprising the amino acid sequence of SEQ ID NO: 3 or 5; or (ii) a modified immunoglobulin-binding domain comprising an amino acid sequence in which one to several amino acid residues have been substituted, deleted, added, and/or inserted to the amino acid sequence of SEQ ID NO: 3 or 5 at amino acid residues other than those at positions 4, 7, and 35;
[5] the method of any one of [1] to [4], wherein the modified immunoglobulin-binding domain has an ability to bind to an IgG antibody comprising the amino acid residue substitutions Q311R and P343R;
[6] the method of any one of [1] to [3], wherein the Protein A-modified ligand is a modified ligand comprising a modified immunoglobulin-binding domain that consists of at least one amino acid sequence selected from the group consisting of SEQ ID NOs: 3, 4, and 5;
[7] the method of any one of [1] to [6], wherein the Protein A-modified ligand is immobilized onto the carrier by any one means selected from the group consisting of (1) to (5) below:
   (1) a method of immobilization onto the carrier through a modified immunoglobulin-binding domain in which 1 to 6 of the amino acid residues at positions 40, 43, 46, 53, 54, and 56 in the C-domain or Z-domain of Protein A are additionally substituted with a lysine residue;
   (2) a method of immobilization onto the carrier through a disulfide bond or a thioether bond by introducing cysteine into the C-terminus of Protein A;
   (3) a method of immobilization onto an amino group-containing immobilization carrier by cyanation of a thiol group;
   (4) a method of immobilizing a multimer of modified immunoglobulin-binding domains having a cysteine residue onto an amino group-containing carrier using 4-(N-maleimidomethyl)cyclohexane-l-carboxylate (SMCC) as a cross-linking agent; and
   (5) a method of immobilization onto the carrier through a plurality of lysine residues added to the C-terminus of a modified immunoglobulin-binding domain in which the lysine residues at positions 42, 49, 50, and 58 of the C-domain variant of Protein A are substituted with amino acids other than lysine, or to a modified immunoglobulin-binding domain in which the lysine residues at positions 49, 50, and 58 of the Z-domain are substituted with amino acids other than lysine;
[8] the method of any one of [1] to [7], wherein the IgG antibody is an IgG antibody additionally comprising one or more amino acid residue substitutions selected from among M428L, N434A, Y436T, Q438R, and S440E in the CH3 region of the IgG antibody;
[9] the method of any one of [1] to [8], wherein the IgG antibody is one in which the CH3 region of the IgG antibody comprises an amino acid sequence selected from the group consisting of SEQ ID NOs: 6 to 11;
[10] the method of any one of [1] to [9], wherein the IgG antibody is one in which the heavy chain constant region of the IgG antibody comprises an amino acid sequence selected from the group consisting of SEQ ID NOs: 12 to 57;
[11] the method of any one of [1] to [10], wherein the pI value of the IgG antibody is 4.0 to 10.0;
[12] the method of any one of [1] to [11], wherein the amount of the Protein A-modified ligand bound to the Fc region of the IgG antibody is 5 times or more compared to the binding ability to unmodified Protein A;
[13] the method of any one of [1] to [12], wherein the method additionally comprises the step of washing the affinity column with a washing solution before step (c);
[14] the method of [13], wherein the washing solution is a combination of a buffer and a salt, and contains, as the buffer, at least one selected from the group consisting of phosphoric acid, acetic acid, citric acid, glycine, and tris hydroxymethyl aminomethane, and as the salt, at least one selected from the group consisting of arginine, sodium chloride, and sodium sulfate;
[15] the method of any one of [1] to [14], wherein the method additionally comprises, after step (c), the step of purifying the IgG antibody by at least one chromatography selected from the group consisting of cation exchange chromatography, anion exchange chromatography, hydrophobic interaction chromatography, multimode chromatography, and hydroxyapatite chromatography;
[16] the method of any one of [1] to [15], wherein step (c) comprises the step of eluting the IgG antibody from the affinity column with an eluting solution containing at least one selected from the group consisting of hydrochloric acid, acetic acid, citric acid, arginine, glycine, and phosphoric acid;
[17] the method of any one of [1] to [16], wherein the antibody is a humanized antibody or a human antibody;
[18] the method of any one of [1] to [17], wherein the antibody is an anti-myostatin antibody, an anti-IL-6 receptor antibody, an anti-IL-6 antibody, an anti-IL-8 antibody, or an anti-IL-31 receptor antibody;
[19] use of an affinity column containing a carrier onto which a Protein A-modified ligand has been immobilized, in the purification of an IgG antibody comprising the amino acid residue substitutions Q311R and P343R, wherein the protein A-modified ligand comprises either or both of an amino acid-substituted C-domain variant and Z-domain of Protein A, wherein the substitution is a substitution which alters any one or more originally present lysine residues at positions 4, 7, and 35 of the C-domain variant or Z-domain to amino acid residues other than lysine, and wherein the protein A-modified ligand is a modified ligand having an ability to bind to the IgG antibody;
[20] a method of producing an IgG antibody having the amino acid residue substitutions Q311R and P343R, wherein the method comprises the following steps of:
   (i) providing a composition containing an IgG antibody comprising the amino acid residue substitutions Q311R and P343R;
   (ii) preparing an affinity column containing a carrier onto which a Protein A-modified ligand is immobilized, wherein the Protein A-modified ligand comprises a modified immunoglobulin-binding domain comprising a modification for substitution of any one or more originally present lysine residues at positions 4, 7, and 35 of the C-domain variant of Staphylococcus Protein A of SEQ ID NO: 1 or the Z-domain of Staphylococcus Protein A of SEQ ID NO: 2 with amino acid residues other than lysine; or a multimer of these modified immunoglobulin-binding domains;
   (iii) loading the composition containing the IgG antibody onto the affinity column of step (ii); and
   (iv) eluting and recovering the IgG antibody from the affinity column loaded with the composition containing the IgG antibody in step (iii).
      Alternatively, the present invention provides:
[1′] a method of purifying an IgG antibody comprising the amino acid residue substitutions Q311R and P343R from a composition containing the antibody, wherein the method comprises the steps of:
   (a) preparing an affinity column containing a carrier onto which a Protein A-modified ligand is immobilized;
   (b) loading the composition containing the IgG antibody onto the affinity column of step (a); and
   (c) eluting and recovering the IgG antibody from the affinity column of step (b), wherein the Protein A-modified ligand contains either or both of an amino acid-substituted C-domain variant and Z-domain of Protein A, wherein the substitution comprises a substitution which alters any one or more originally present lysine residues at positions 4, 7, and 35 of the C-domain variant or Z-domain to amino acid residues other than lysine, and is a Protein A-modified ligand having an activity to bind to the IgG antibody.

### Advantageous Effect of Invention

By the present invention, even pI-modified antibodies that cannot be successfully purified by a common Protein A column can be purified easily and efficiently.

### BRIEF DESCRIPTION OF DRAWINGS

FIG. 1 shows the results of measuring the dynamic binding capacity (DBC) of an antibody that does not contain a modification in the Fc region in each Protein A-immobilized resin. In the figure, the vertical axis shows DBC (g/L resin), and the horizontal axis shows residence time (minutes).

FIG. 2-1 shows the result (real-time binding curve) of evaluating the binding affinity between the ligand (structure represented by Formula (1′)) for AF-rProtein A HC-650F and an antibody, using the BLItz (registered trademark) evaluation system (ForteBio).

FIG. 2-2 shows the result (real-time binding curve) of evaluating the binding affinity between the ligand for MabSelect SuRe and an antibody, using the BLItz (registered trademark) evaluation system (ForteBio).

### DESCRIPTION OF EMBODIMENTS

Hereinbelow, the present invention will be described in detail.

The present invention relates to methods of purifying a composition containing a pI-modified antibody having an increased isoelectric point (pI). Specifically, the present invention relates to methods of purifying an IgG antibody comprising the amino acid residue substitutions Q311R and P343R from a composition containing the antibody, wherein the method comprises the following steps of:
(a) preparing an affinity column containing a carrier onto which a Protein A-modified ligand is immobilized, wherein the Protein A-modified ligand comprises: a modified immunoglobulin-binding domain containing a modification for substitution of any one or more originally present lysine residues at positions 4, 7, and 35 of the C-domain variant of Staphylococcus Protein A of SEQ ID NO: 1 or the Z-domain of Staphylococcus Protein A of SEQ ID NO: 2 with amino acid residues other than lysine; or a multimer of these modified immunoglobulin-binding domains;
(b) loading the composition containing the IgG antibody onto the affinity column of step (a); and
(c) eluting and recovering the IgG antibody loaded in step (b).

The IgG antibodies comprising the amino acid residue substitutions Q311R and

P343R with regard to the CH2 and CH3 regions in the present invention are antibodies in which both glutamine (Q) at position 311 and proline (P) at position 343 of the CH2 and CH3 regions (according to EU numbering) in the parent Fc region have been modified to arginine (R). In general, the CH2 region corresponds to the amino acids at positions 231 to 340, and the CH3 region corresponds to the amino acids at positions 341 to 447 (according to EU numbering) within the hinge region. The “parent Fc region” in the present application refers to an Fc region before the introduction of the amino acid modifications described in the present specification. Preferred examples of the parent Fc region include Fc regions derived from natural antibodies. Antibodies can be derived from humans or monkeys (e.g., cynomolgus monkeys, rhesus monkeys, marmosets, chimpanzees, or baboons). Natural antibodies may comprise naturally occurring mutations. Multiple allotype sequences of IgG due to genetic polymorphisms are described in “Sequences of Proteins of Immunological Interest”, NIH Publication No. 91-3242, all of which can be used in the present invention. In particular, for human IgG1, the amino acid sequence at positions 356-358 (EU numbering) can be either DEL or EEM. Preferred examples of the parent Fc region include an Fc region derived from a heavy chain constant region of human IgG1 (SEQ ID NO: 58), human IgG2 (SEQ ID NO: 59), human IgG3 (SEQ ID NO: 60) and human IgG4 (SEQ ID NO: 61). Another preferred example of the parent Fc region is an Fc region derived from the heavy chain constant region SG1 (SEQ ID NO: 62). Further, the parent Fc region may be an Fc region prepared by adding amino acid modifications other than the amino acid modifications described in the present specification to an Fc region derived from the natural antibody.

With respect to the antibody in the present invention, amino acid modifications carried out for other purposes may be combined with the antibodies used in the present invention. For example, amino acid substitutions that enhance FcRn-binding activity (Hinton et al., J. Immunol. 176(1): 346-356 (2006); Dall'Acqua et al., J. Biol. Chem. 281(33): 23514-23524 (2006); Petkova et al., Intl. Immunol. 18(12): 1759-1769 (2006); Zalevsky et al., Nat. Biotechnol. 28(2): 157-159 (2010); WO 2006/019447; WO 2006/053301; and WO 2009/086320), and amino acid substitutions for improving antibody heterogeneity or stability (WO 2009/041613) may be added. Alternatively, amino acid modifications applied to polypeptides having properties that promote antigen clearance as described in WO 2011/122011, WO 2012/132067, WO 2013/046704, or WO 2013/180201, polypeptides having specific binding properties to target tissues as described in WO 2013/180200, or polypeptides having the property of repeatedly binding to multiple antigen molecules as described in WO 2009/125825, WO 2012/073992, or WO 2013/047752 may be combined with the antibodies used in the present invention. The amino acid modifications disclosed in EP1752471 and EP1772465 may be combined with the antibodies used in the present invention for the purpose of imparting binding ability to other antigens Amino acid modifications that lower the pI of the constant region (WO 2012/016227) may be combined with the antibodies used in the present invention for the purpose of increasing plasma retention Amino acid modifications that increase the pI of the constant region (WO 2014/145159) may be combined for the purpose of promoting uptake into cells Amino acid modifications that increase the pI of the constant region (Japanese Patent Application Nos. 2015-021371 and 2015-185254) may be combined for the purpose of promoting the elimination of a target molecule from plasma.

In the present invention, amino acid modification means any substitution, deletion, addition, insertion, and modifications, or combinations thereof. In the present invention, an amino acid modification can be paraphrased as an amino acid mutation.

The antibodies used in the present invention are more preferably IgG antibodies which further comprise one or more amino acid residue substitutions selected from M428L, N434A, Y436T, Q438R, and S440E in the CH3 region, and even more preferably, they are IgG antibodies which comprise in the CH3 region two or more amino acid residue substitutions selected from M428L, N434A, Y436T, Q438R, and S440E. Further preferably, they include IgG antibodies where the CH3 region comprises an amino acid sequence selected from the group consisting of SEQ ID NOs: 6 to 11, and IgG antibodies where the heavy chain constant region comprises an amino acid sequence selected from the group consisting of SEQ ID NOs: 12 to 57.

The antibodies used in the present invention are usually not particularly limited as long as they bind to a desired antigen, and may be polyclonal antibodies or monoclonal antibodies.

Monoclonal antibodies used in the present invention include not only monoclonal antibodies derived from animals such as humans, mice, rats, hamsters, rabbits, sheep, camels and monkeys, but also artificially-modified recombinant antibodies such as chimeric antibodies, humanized antibodies, bispecific antibodies. Furthermore, recombinant antibodies where an antibody's constant region and the like are artificially modified in order to alter the physical properties of antibody molecules for the purpose of improving blood retention and in vivo kinetics (specifically, to modify the isoelectric point (pI), affinity to Fc receptors, etc.) are also included.

The immunoglobulin class of the antibodies used in the present invention is not particularly limited, and IgGs such as IgG1, IgG2, IgG3, and IgG4 may be used. Preferred IgGs in the present invention are IgG1, IgG2, and IgG4, especially if the Fc region is of human origin.

The antibodies used in the present invention can also be used as pharmaceutical compositions, and can be administered using any known method including parenteral administration, intrapulmonary administration, and nasal administration, and if desired for topical treatment, intralesional administration. Parenteral injections include intramuscular, intravenous, intraperitoneal, and subcutaneous administration.

When an antibody used in the present invention is used as a pharmaceutical composition, a product that contains the pharmaceutical composition and equipment useful for treatment, prevention, and/or diagnosis is provided. The product includes a container, a label on the container and a package insert attached to the container. Preferred containers include, for example, bottles, vials, syringes, IV solution bags, and the like. Containers may be made of various materials such as glass and plastic, and silicon-free syringes and the like can also be used.

The antibodies used in the present invention described above can be produced by a method well known in the art. A hybridoma that produces a monoclonal antibody can be produced as follows, basically using a known technique. More specifically, a desired antigen or cells expressing a desired antigen is/are used as a sensitizing antigen, which is used for immunization according to a normal immunization method, and the obtained immune cells are fused with known parent cells by a normal cell fusion method, and a monoclonal antibody can be produced by screening for monoclonal antibody-producing cells (hybridomas) by a conventional screening method. The hybridoma can be produced, for example, according to the method of Milstein et al. (Kohler, G. and Milstein, C., Methods Enzymol. (1981) 73: 3-46) or such. When the immunogenicity of the antigen is low, the immunization can be done by coupling the antigen to a macromolecule having immunogenicity such as albumin.

In addition, it is possible to use a recombinant antibody produced by cloning an antibody gene from a hybridoma, inserting it into an appropriate vector, introducing it into a host, and producing it using a gene recombination technique (see, for example, Carl, A. K. Borrebaeck, James, W. Larrick, THERAPEUTIC MONOCLONAL ANTIBODIES, Published in the United Kingdom by MACMILLAN PUBLISHERS LTD., 1990). Specifically, cDNA of the variable region (V region) of the antibody is synthesized from the mRNA of the hybridoma using a reverse transcriptase. Once the DNA encoding the V region of the antibody of interest is obtained, it is ligated with a DNA encoding the desired antibody constant region (C region) and inserted into an expression vector. Alternatively, the DNA encoding the V region of the antibody may be inserted into an expression vector containing the DNA of the antibody C region. They are inserted into an expression vector so that it is expressed under the control of an expression control region, for example, an enhancer or a promoter. The host cells can then be transformed with this expression vector to express the antibody.

In the present invention, a recombinant antibody that has been artificially modified to reduce heterologous antigenicity to humans or such, for example, a chimeric antibody, a humanized antibody, or the like can be used. These modified antibodies can be produced using known methods. A chimeric antibody is an antibody consisting of the variable regions of the heavy and light chains of a non-human mammal antibody, for example, a mouse antibody, and the constant regions of the heavy and light chains of a human antibody. The antibody can be obtained by ligating a DNA encoding the variable region of the mouse antibody with a DNA encoding the constant region of the human antibody, inserting the ligated DNA into an expression vector, introducing into a host to produce the antibody therein.

A humanized antibody, also called a reshaped human antibody, is obtained by transplanting the complementarity determining regions (CDRs) of a non-human mammal antibody, such as a mouse antibody, into the complementarity determining regions of a human antibody, and the general gene recombination technique for this is also known. Specifically, a DNA sequence designed to connect the CDRs of the mouse antibody and the framework regions (FRs) of the human antibody is synthesized by the PCR method from several oligonucleotides prepared so as to have overlapping portions at their terminal portions. The resulting DNA is ligated with a DNA encoding the human antibody constant region, inserted the DNA into an expression vector, and introduced into a host for antibody production (see EP 239400, WO 96/02576). The FRs of the human antibody linked via CDRs are selected so that the complementarity determining regions form a good antigen-binding site. If desired, the amino acids in the framework regions of the variable regions of the antibody may be substituted so that the complementarity determining regions of the reshaped human antibody form the appropriate antigen-binding site (Sato, K. et al., Cancer Res. (1993) 53, 851-856).

The following techniques are known as examples for substituting amino acids of an antibody in order to improve the activity, physical properties, pharmacokinetics, safety, and such of the antibody, and the antibodies used in the present invention also include such antibodies with amino acid substitutions (including deletions and additions).

The following have been reported as techniques for substituting amino acids in the variable regions of IgG antibodies:
humanization (Tsurushita N, Hinton PR, Kumar S, Design of humanized antibodies: from anti-Tac to Zenapax., Methods. 2005 May; 36(1): 69-83.);
affinity maturation by amino acid substitutions of complementarity determination regions (CDRs) to enhance binding activity (Rajpal A., Beyaz N, Haber L, Cappuccilli G, Yee H, Bhatt RR, Takeuchi T, Lerner RA, Crea R, A general method for greatly improving the affinity of antibodies by using combinatorial libraries., Proc Natl Acad Sci USA. 2005 Jun. 14; 102(24): 8466-71.); and
improvement of physicochemical stability by amino acid substitutions in frameworks (FRs) (Ewert S, Honegger A, Pluckthun A., Stability improvement of antibodies for extracellular and intracellular applications: CDR grafting to stable frameworks and structure-based framework engineering., Methods. 2004 October; 34(2): 184-99. Review).

Further, as techniques for substituting amino acids in the Fc region of an IgG antibody, techniques that enhance antibody-dependent cellular cytotoxicity (ADCC) activity and/or complement-dependent cellular cytotoxicity (CDC) activity are known (Kim S J, Park Y, Hong H J., Antibody engineering for the development of therapeutic antibodies., Mol Cells. 2005 Aug. 31; 20(1): 17-29. Review.). Also reported is a technique for amino acid substitutions in the Fc region, which not only enhances such effector functions but also improves the blood half-life of antibodies (Hinton PR, Xiong J M, Johlfs M G, Tang M T, Keller S, Tsurushita N, An engineered human IgG1 antibody with longer serum half-life., J. Immunol. 2006 Jan. 1; 176(1): 346-56.; Ghetie V, Popov S, Borvak J, Radu C, Matesoi D, Medesan C, Ober RJ, Ward ES, Increasing the serum persistence of an IgG fragment by random mutagenesis., Nat Biotechnol. 1997 July; 15(7): 637-40.). Furthermore, various amino acid substitution techniques in the constant region for the purpose of improving the physical properties of antibodies are also known (WO 09/41613).

Methods for obtaining human antibodies are also known. For example, it is possible to obtain a desired human antibody with binding activity to an antigen by immunizing human lymphocytes in vitro with the desired antigen or cells expressing the desired antigen, and fusing the immunized lymphocytes with human myeloma cells, such as U266 (see JP-A (Kohyo) H01-59878). In addition, a desired human antibody can be obtained by immunizing transgenic animals having the complete repertoire of human antibody genes with an antigen (see WO 93/12227, WO 92/03918, WO 94/02602, WO 94/25585, WO 96/34096, WO 96/33735). Technologies for obtaining a human antibody by panning using a human antibody library are also known. For example, a phage that binds to an antigen can be selected by expressing the variable region of a human antibody as a single-chain antibody (scFv) on the surface of the phage by the phage display method. By analyzing the genes of the selected phage, the DNA sequence encoding the variable region of the human antibody that binds to the antigen can be determined. Once the DNA sequence of scFv that binds to the antigen is clarified, a suitable expression vector containing the sequence can be prepared and the human antibody can be obtained. These methods are already well known and WO 92/01047, WO 92/20791, WO 93/06213, WO 93/11236, WO 93/19172, WO 95/01438, WO 95/15388 can be referred to. Antibodies used in the present invention also include such human antibodies.

When an antibody gene is once isolated and introduced into a suitable host to prepare an antibody, a suitable combination of host and an expression vector can be used. When eukaryotic cells are used as host, animal cells, plant cells, and fungal cells can be used. Animal cells including (1) mammalian cells, for example, CHO, COS, myeloma, BHK (baby hamster kidney), HeLa, Vero; (2) amphibian cells, for example, Xenopus oocytes; and (3) insect cells, for example, sf9, sf21, Tn5, and such are known. As plant cells, cells derived from the genus Nicotiana, for example, Nicotiana tabacum, are known, and these cells may be callus cultured. Known fungal cells include yeasts such as the genus Saccharomyces, for example, Saccharomyces cerevisiae, and filamentous fungi, such as the genus Aspergillus, for example, Aspergillus niger. When using prokaryotic cells, there are production systems that use bacterial cells. E. coli and Bacillus subtilis are known as bacterial cells. Antibodies can be obtained by introducing the target antibody genes into these cells by transformation and culturing the transformed cells in vitro.

Antibodies linked to various molecules such as polyethylene glycol (PEG) and cytotoxic agents can also be used as antibody modification products (Farmaco. 1999 Aug 30; 54(8): 497-516., Cancer J. 2008. May-June; 14(3): 154-69.). These antibody modification products are also encompassed by the antibodies used in the present invention. Such antibody modification products can be obtained by chemically modifying an antibody. These methods have already been established in this field.

Antibodies used in the present invention include anti-tissue factor antibodies, anti-IL-6 receptor antibodies, anti-IL-6 antibodies, anti-HM1.24 antigen monoclonal antibodies, anti-parathyroid hormone-related peptide antibodies (anti-PTHrP antibodies), anti-glypican-3 antibodies, anti-ganglioside GM3 antibodies, anti-TPO receptor agonist antibodies, coagulation Factor VIII function-substituting antibodies, anti-IL31 receptor antibodies, anti-HLA antibodies, anti-AXL antibodies, anti-CXCR4 antibodies, anti-NR10 antibodies, and bispecific antibodies that recognize Factor IX(a) and Factor X, but are not limited thereto.

Further, the pI values of the antibodies used in the present invention are preferably from 4.0 to 10.0, more preferably from 5.0 to 9.5, and still more preferably from 6.0 to 9.0. The pI values are elevated compared to the pI value of the IgG antibody before the amino acid modification. The isoelectric point of an IgG antibody or the like can be evaluated by a known analysis method such as isoelectric focusing.

The amount of binding between a Protein A-modified ligand in the present invention and the Fc region of the IgG antibody to be purified in the present invention is preferably 5 times or more, more preferably 10 times or more, as compared to the amount of binding to unmodified Protein A. The binding amount referred to here is not particularly limited to the method of measurement, and an example thereof includes the method of measuring the dynamic binding capacity described in the Examples herein.

Commonly-used Protein A columns specifically include, for example, HiTrap MabSelect SuRe (manufactured by GE Healthcare, trade name), Amsphere A3 (manufactured by JSR Life Sciences, registered trademark), MiniChrom Column Eshmuno A (manufactured by Merck Millipore, registered trademark), MabSpeed rP202 (manufactured by Mitsubishi Chemical, registered trademark), and KanCap Pre-packaged Column (manufactured by Kaneka, trade name)

The Protein A affinity column used in the present invention includes an affinity column containing a carrier onto which a Protein A-modified ligand has been immobilized, wherein the Protein A-modified ligand comprises: a modified immunoglobulin-binding domain comprising a modification for substitution of any one or more originally present lysine residues at positions 4, 7, and 35 of the C-domain variant or Z-domain with amino acid residues other than lysine, and also has a binding ability to an IgG antibody comprising the amino acid residue substitutions Q311R and R343R; or a multimer of these modified immunoglobulin-binding domains. This Protein A-modified ligand is characterized in that, when immobilized onto an insoluble carrier via its own amino groups, the orientation for maintaining the affinity for immunoglobulin is improved as compared with the unmodified molecule, through the modification of substitution for any one or more lysine residues at positions 4, 7 and 35 of the C-domain variant (SEQ ID NO: 1) or Z-domain (SEQ ID NO: 2) with other amino acid residues, the C-domain variant being a domain in which the glycine residue at position 29 of the amino acid sequence of the C-domain of Protein A has been substituted with an alanine residue.

In the present invention, the “immunoglobulin-binding domain” refers to a functional unit of a polypeptide having an immunoglobulin-binding activity by itself, and the “modified immunoglobulin-binding domain” is a domain in which a modification has been added to the original immunoglobulin-binding domain. The “ligand” refers to a molecule having the property of binding to a specific molecule by a specific affinity, and in the present invention, refers to an immunoglobulin-binding protein that can selectively bind to immunoglobulins. The “Protein A-modified ligand” refers to an immunoglobulin-binding protein comprising a modified immunoglobulin-binding domain in which the binding domain of Protein A has been modified. Here, in the present specification, the “modified immunoglobulin-binding domain” and the “Protein A-modified ligand” are collectively referred to as “modified protein”.

The modified immunoglobulin-binding domain used in the present invention can contain an amino acid sequence in which, in the C-domain variant (SEQ ID NO: 1) or in the Z-domain (SEQ ID NO: 2), one or more lysine residues at positions 4, 7, and 35 are additionally substituted with other amino acid residues. For example, of the positions 4, 7, and 35, it is desirable that two or more lysine residues, preferably three lysine residues, are substituted with other amino acid residues.

The type of amino acid after substitution at any one or more of positions 4, 7, and 35 of the C-domain variant (SEQ ID NO: 1) or Z-domain (SEQ ID NO: 2) is not particularly limited, but it is preferably alanine, glutamine, asparagine, valine, serine, threonine, histidine, tyrosine, or arginine, and more preferably alanine, threonine, or arginine.

More specifically, the type of amino acid after substitution at position 4 of the C-domain variant (SEQ ID NO: 1) or Z-domain (SEQ ID NO: 2) is preferably glutamic acid, isoleucine, arginine, alanine, valine, serine, threonine, or histidine, and more preferably alanine.

The type of amino acid after substitution at position 7 of the C-domain variant (SEQ ID NO: 1) or Z-domain (SEQ ID NO: 2) is preferably tyrosine, phenylalanine, glutamine, leucine, isoleucine, proline, threonine, alanine, valine, serine, arginine, or histidine, and more preferably threonine.

The type of amino acid after substitution at position 35 of the C-domain variant (SEQ ID NO: 1) or Z-domain (SEQ ID NO: 2) is preferably arginine, glutamine, asparagine, or tyrosine, and more preferably arginine.

In addition to the above modifications, the C-domain variant (SEQ ID NO: 1) or Z-domain (SEQ ID NO: 2) may additionally have one to several amino acids substituted to the extent that it has the ability to bind to an IgG antibody comprising the amino acid residue substitutions Q311R and R343R. In particular, in the C-domain variant (SEQ ID NO: 1) or Z-domain (SEQ ID NO: 2), it is preferred that the number of lysines contained as constituent amino acids is preferably small, and it is desirable that in addition to the above modifications, 1 to 4, preferably 3 or 4, or more preferably 4 of the originally present lysine residues at positions 42, 49, 50 and 58 are substituted with amino acid residues other than lysine.

The type of amino acid after substitution at any one or more of positions 42, 49, 50, and 58 of the C-domain variant (SEQ ID NO: 1) or Z-domain (SEQ ID NO: 2) is not particularly limited, but is preferably alanine, glutamine, asparagine, valine, serine, threonine, histidine, tyrosine, or arginine, and more preferably alanine or arginine.

More specifically, if the originally present lysine residue at position 42 of the C-domain variant (SEQ ID NO: 1) or Z-domain (SEQ ID NO: 2) is substituted with another amino acid residue, the type of amino acid after the substitution is preferably alanine, valine, serine, threonine, or histidine, and more preferably alanine.

If the originally present lysine residue at position 49 of the C-domain variant (SEQ ID NO: 1) or Z-domain (SEQ ID NO: 2) is substituted with another amino acid residue, the type of amino acid after the substitution is preferably arginine, glutamine, asparagine, or tyrosine, and more preferably arginine.

If the originally present lysine residue at position 50 of the C-domain variant (SEQ ID NO: 1) or Z-domain (SEQ ID NO: 2) is substituted with another amino acid residue, the type of amino acid after the substitution is preferably arginine, glutamine, asparagine, or tyrosine, and more preferably arginine.

If the originally present lysine residue at position 58 of the C-domain variant (SEQ ID NO: 1) or Z-domain (SEQ ID NO: 2) is substituted with another amino acid residue, the type of amino acid after the substitution is preferably arginine, glutamine, asparagine, or tyrosine, and more preferably arginine.

Furthermore, in the modified immunoglobulin-binding domain in the present invention, in addition to the above modifications, the aspartic acid residue at position 37 of the C-domain variant (SEQ ID NO: 1) or Z-domain (SEQ ID NO: 2) may be substituted with an amino acid residue other than aspartic acid. This modification improves the chemical stability under acidic pH conditions as compared to an unmodified molecule.

If the original aspartic acid residue at position 37 of the C-domain variant (SEQ ID NO: 1) or Z-domain (SEQ ID NO: 2) is substituted with another amino acid residue, the type of amino acid after the substitution is not particularly limited, but alanine, glutamic acid, serine, threonine, leucine, or isoleucine is preferred.

A suitable example of the amino acid sequence of the modified immunoglobulin-binding domain used in the present invention includes the amino acid sequence of SEQ ID NO: 3 or 5. The amino acid sequence of SEQ ID NO: 3 is an amino acid sequence in which position 4 in the amino acid sequence of SEQ ID NO: 1 has been substituted with an alanine residue, position 7 with a threonine residue, and position 35 with an arginine residue. The amino acid sequence of SEQ ID NO: 5 is an amino acid sequence in which position 4 in the amino acid sequence of SEQ ID NO: 1 has been substituted with an alanine residue, position 7 with a threonine residue, position 35 with an arginine residue, position 42 with an alanine residue, position 49 with an arginine residue, position 50 with an arginine residue, and position 58 with an arginine residue.

Alternatively, in addition to the above sequences, suitable examples of amino acid sequences of the modified immunoglobulin-binding domain in the present invention can include the following amino acid sequences:
[1] in the C-domain variant defined by SEQ ID NO: 1, the lysine at position 35 is substituted with glutamine or arginine;
[2] in the C-domain variant defined by SEQ ID NO: 1, the amino acid residues at positions 40, 43, 46, and 53 are substituted with lysine, and the lysine at position 35 is substituted with arginine or valine;
[3] additionally in the amino acid sequence of [2], the lysine at position 7 is substituted with tyrosine, phenylalanine, threonine, arginine, glutamine, valine, leucine, isoleucine, histidine, alanine, or proline;
[4] additionally in the amino acid sequence of [2] or [3], the lysine at position 4 is substituted with alanine;
[5] in the C-domain variant defined by SEQ ID NO: 1, the amino acid residues at positions 40, 43, 46, and 53 are substituted with lysine, and the lysine at position 4 is substituted with valine, isoleucine, glutamic acid, or arginine;
[6] in the C-domain variant defined by SEQ ID NO: 1, the lysine at position 42 is substituted with alanine, the lysines at positions 49, 50, and 58 are substituted with arginine, and additionally the lysine at position 4 is substituted with valine, isoleucine, glutamic acid or arginine; and
[7] additionally in the amino acid sequence of [5] or [6], the lysines at positions 7 and 35 are substituted with arginine.

When the Protein A-modified ligand is a multimer having an immunoglobulin-binding domain as the constituent unit, it is sufficient to contain one or more immunoglobulin-binding domains comprising the above modification(s). The number of units of the immunoglobulin binding domain contained in the multimer is, for example, 2 to 10, preferably 2 to 8, more preferably 4 to 7, and even more preferably 6. The multimer may contain immunoglobulin-binding domains that do not contain the above-mentioned modification(s), as long as it contains one or more immunoglobulin-binding domains that contain the above-mentioned modification(s). In the multimer, the ratio of immunoglobulin-binding domains containing the above-mentioned modification(s) to the total number of immunoglobulin-binding domains contained as constituent units is preferably 50% or more, and more preferably 100% (that is, all of the immunoglobulin-binding domains contained as constituent units have the above-mentioned modification(s)).

When the Protein A-modified ligand is a multimer having an immunoglobulin-binding domain as the constituent unit, the immunoglobulin-binding domain arranged at the first and/or second from the N-terminal or C-terminal side of the multimer may be substituted with an amino acid residue that can covalently bind to the carrier (e.g., lysine residue) to facilitate immobilization onto the carrier. For example, immobilization onto the carrier becomes easier when at least 1, preferably 2 to 6, more preferably 3 or 4, and even more preferably 4 originally present amino acid residues from among those at positions 40, 43, 46, 53, 54, and 56 in the C-domain variant (SEQ ID NO: 1) or Z-domain (SEQ ID NO: 2) that is arranged at the first and/or second from the N-terminal or C-terminal side of the multimer are substituted with lysine residues.

Further, in the immunoglobulin-binding domain arranged at the first and/or second from the N-terminal or C-terminal side of the multimer, one or more lysine residues at positions 4, 7, and 35 in the C-domain variant (SEQ ID NO: 1) or Z-domain (SEQ ID NO: 2) may not be substituted with other amino acid residues, but one or more of these lysine residues may be substituted with other amino acid residues. An example of an immunoglobulin-binding domain arranged at the first and/or second from the N-terminal or C-terminal side of the multimer includes the amino acid sequence of SEQ ID NO: 4. In the amino acid sequence of SEQ ID NO: 4, position 4 in the amino acid sequence of SEQ ID NO: 1 has been substituted with an alanine residue, position 7 with a threonine residue, position 35 with an arginine residue, position 40 with a lysine residue, position 43 with a lysine residue, position 46 with a lysine residue, and position 53 with a lysine residue.

An example of the above multimer includes a modified Protein A ligand represented by the following Formula (1).

(R1)ₙ-(R2)ₘ or (R2)ₘ-(R1)ₙ (1)

In Formula (1), the left end is the N-terminus and the right end is the C-terminus.

In Formula (1), “n” is an integer of 1 or more and 9 or less, preferably an integer of 1 or more and 7 or less, more preferably an integer of 3 or more and 6 or less, and further preferably 5. “m” is an integer of 1 or 2, and preferably 1. The total number of domains “n+m” is 2 to 10, preferably 2 to 8, more preferably 4 to 7, and further preferably 6.

In Formula (1), (R1) is a modified immunoglobulin-binding domain in which one or more (preferably all) lysine residues at positions 4, 7, and 35 of the C-domain variant (SEQ ID NO: 1) or Z-domain (SEQ ID NO: 2) have been substituted with amino acid residues other than a lysine residue. It is preferred that in (R1), in addition to the substitutions at positions 4, 7, and 35 mentioned above, any one or more (preferably all) lysine residues at positions 42, 49, 50, and 58 have been substituted with amino acid residues other than lysine. The “n” number of (R1) may all have the same amino acid sequence, or may have amino acid sequences different from each other.

In Formula (1), (R2) is an immunoglobulin-binding domain in which one or more (preferably all) amino acid residues at positions 40, 43, 46, 53, 54, and 56 of the C-domain variant (SEQ ID NO: 1) or Z-domain (SEQ ID NO: 2) have been substituted with a lysine residue. It is preferred that in (R2), in addition to any one or more of the substitutions at positions 40, 43, 46, 53, 54, and 56, any one or more (preferably all) lysine residues at positions 4, 7, and 35 have been substituted with amino acid residues other than lysine. When “m” is 2, the two (R2) may both have the same amino acid sequence, but may also have different sequences from each other.

A suitable example of the modified Protein A ligand represented by the above Formula (1) includes “n” is 5; “m” is 1; (R1) is a modified immunoglobulin-binding domain consisting of the amino acid sequence of SEQ ID NO: 5 (an amino acid sequence where position 4 in SEQ ID NO: 1 is substituted with an alanine residue, position 7 with a threonine residue, position 35 with an arginine residue, position 42 with an alanine residue, position 49 with an arginine residue, position 50 with an arginine residue, and position 58 with an arginine residue); and (R2) is a modified immunoglobulin-binding domain consisting of the amino acid sequence of SEQ ID NO: 4 (an amino acid sequence where position 4 in SEQ ID NO: 1 is substituted with an alanine residue, position 7 with a threonine residue, position 35 with an arginine residue, position 40 with a lysine residue, position 43 with a lysine residue, position 46 with a lysine residue, and position 53 with a lysine residue).

A Protein A-modified protein of the present invention can be produced by using known genetic engineering techniques described in, for example, Current Protocols in Molecular Biology by Frederick M. Ausbel et al. More specifically, the protein can be obtained from cultured cells in a large amount at a low cost by transforming a host such as Escherichia coli with an expression vector including a nucleotide sequence encoding a target modified protein and culturing the cells in an appropriate liquid medium. Specifically, since one immunoglobulin-binding domain of Protein A is a small protein consisting of about 60 amino acids, a target expression vector can be obtained by, for example, dividing a DNA encoding a desired amino acid sequence into synthetic oligonucleotides consisting of several tens of bases, synthesizing them, ligating them by a ligation reaction with DNA ligase, and inserting them into a plasmid vector.

At that time, for the purpose of efficiently expressing the protein in E. coli, one skilled in the art usually employs a nucleotide sequence using the optimum codons of E. coli. Further, any domain of Protein A may be employed as the amino acid sequence of an unmodified immunoglobulin-binding domain, but among the five originally existing domains, a C-domain having many lysine residues at positions 39 onwards is preferably used. Alternatively, the Z-domain sequence that has often been used as an affinity ligand for immunoglobulins may be utilized, but it is most preferable to employ the sequence of the C-domain variant (shown in SEQ ID NO: 1 in Sequence Listing) where the glycine residue at position 29 has been substituted with an alanine residue, which has already been known to increase chemical stability (Nilsson B. et. al., Protein Engineering, 1(2), pp.107-113). Mutations in the DNA sequence for achieving the target amino acid substitutions can be easily introduced into intended sites by using a method such as the overlap extension method using an unmodified clone DNA as a template and using, as primers, synthetic oligo DNAs which incorporate mismatched base pairs for the polymerase chain reaction, and the cassette mutation method. Furthermore, in the case of using a Protein A-derived immunoglobulin-binding protein as an affinity chromatography ligand for an immunoglobulin, a multimeric protein obtained by ligating two or more, desirably about four immunoglobulin-binding domains has been conventionally produced and used. For the immunoglobulin-binding protein obtained by the present invention, it is preferable to produce and use a multimeric protein obtained by ligating two or more, preferably two to ten, more preferably four to seven, and even more preferably six immunoglobulin-binding domains. A cDNA encoding such a multimeric protein can be easily prepared by linking the intended number of cDNAs each encoding one immunoglobulin-binding domain in series. A multimeric protein in which two or more of immunoglobulin-binding domain units are linked can be easily produced by using thus prepared cDNA inserted into an appropriate expression plasmid.

Any vectors such as plasmids, phages, or viruses that can replicate itself in host cells can be used as the expression vector to be inserted with a nucleotide sequence encoding the modified protein of the present invention. For example, commercially available expression vectors include pQE system vectors (QIAGEN), pDR540, pRIT2T (GE Healthcare Bioscience Co., Ltd.), pET system vectors (Merck Co., Ltd.). The expression vector is preferably used by selecting an appropriate combination with the host cell. For example, when E. coli is used as a host cell, preferred examples include a combination of a pET system vector and the BL21 (DE3) E. coli strain and a combination of the pDR540 vector and the JM109 E. coli strain.

The modified protein of the present invention can be recovered in a soluble fraction by collecting cultured cells by centrifugation or the like and homogenizing them by a treatment using ultrasonic waves, French press, or the like. Purification of the modified protein can be performed by appropriately combining known separation/purification techniques. Specifically, techniques include separation techniques such as the salting-out, dialysis, and ultrafiltration; and purification methods such as hydrophobic chromatography, gel filtration chromatography, ion exchange chromatography, affinity chromatography, and reverse-phase chromatography.

Examples of an insoluble carrier for binding to the modified protein of the present invention as an affinity ligand for immunoglobulin include natural polymer materials such as chitosan and dextran, and synthetic polymers such as vinyl polymers, highly crosslinked agarose, and polyimide. In another embodiment, the carrier may be inorganic carriers such as silica. In general, a ligand protein is immobilized onto a carrier with a coupling agent such as cyanogen bromide, epichlorohydrin, N-hydroxy succinimide, tosyl/tresyl chloride, carbodiimide, glutaraldehyde, hydrazine, or a carboxyl- or thiol-activated carrier. Such coupling reactions are well known in the art and are widely described in literatures (for example, Jansson, J. C. and Ryden, L., “Protein purification”, 2nd Edition, pp. 375-442, ISBN 0-471-18626-0). The ligand protein of the present invention is characterized in that the protein binds to a carrier via a plurality of amino groups arranged so that orientation of the ligand can be spatially controlled. For immobilization of the protein, a carrier having an active group that can form a covalent bond by a reaction with an amino group of the protein, such as a tresyl group, an epoxy group, a carboxyl group, and a formyl group can be used. Examples of commercially available carriers include TOYOPEARL AF-Tresyl-650, TOYOPEARL AF-Epoxy-650, TOYOPEARL AF-Carboxy-650, TOYOPEARL AF-Formyl-650 (all from Tosoh Corporation), NHS-activated Sepharose, cyanogen bromide-activated Sepharose, and epoxy-activated Sepharose (all from GE Healthcare Bioscience Co., Ltd.).

As the Protein A affinity column used in the present invention, the above Protein A-modified ligand may be immobilized by any means. For example, it can be immobilized by the following means:
(1) a method of additionally substituting 1 to 6 of the amino acid residues at positions 40, 43, 46, 53, 54 and 56 in the C-domain variant or Z-domain of Protein A with a lysine residue and immobilizing it onto the carrier through the substituted lysine residue(s);
(2) a method of immobilization onto the carrier through a disulfide bond or a thioether bond by introducing cysteine into the C-terminus of Protein A;
(3) a method of immobilization onto an amino group-containing immobilization carrier by cyanation of a thiol group;
(4) a method of immobilizing a multimer of modified immunoglobulin-binding domains having a cysteine residue onto an amino group-containing carrier using 4-(N-maleimidomethyl)cyclohexane- 1-carboxylate (SMCC) as a cross-linking agent; and
(5) a method of immobilization onto the carrier through a plurality of (for example, five) lysine residues added to the C-terminus of a modified immunoglobulin-binding domain in which lysine residues at positions 42, 49, 50, and 58 of the C-domain variant of Protein A have been substituted with amino acids other than lysine, or to the C-terminus of a modified immunoglobulin-binding domain in which lysine residues at positions 49, 50, and 58 of the Z-domain of Protein A with amino acids other than lysine.
   The above immobilization methods can be carried out by the usual methods.

Preferred method is the method of additionally substituting 1 to 6 of the amino acid residues at positions 40, 43, 46, 53, 54 and 56 in the C-domain variant or Z-domain of Protein A with a lysine residue and immobilizing it onto the carrier through the substituted lysine residue(s).

The Protein A affinity columns used in the present invention specifically include AF-rProtein A HC-650F (manufactured by Tosoh), which is an affinity resin in which an Fc-binding ligand (recombinant of modified Protein A) has been coupled to the synthetic polymer carrier TOYOPEARL HW-56.

The thus prepared carrier onto which a Protein A-modified ligand has been immobilized can be packed in a column to prepare an affinity column (step a). Next, the prepared affinity column is loaded with a composition containing an IgG antibody comprising modified amino acid sequence in the CH2 region and CH3 region (step b). In the present invention, the composition containing an IgG antibody refers to, for example, a culture of IgG antibody-expressing cells or supernatants thereof. In general, a culture constitutes a complex composition composed of various components such as metabolites of cells as well as various nutrients necessary for culturing. In order to highly purify the target IgG antibody to the purity required for pharmaceutical raw materials from that, it is necessary to determine the purification conditions suitable for the target IgG antibody. The present invention revealed that, to purify an IgG antibody comprising the amino acid residue substitutions Q311R and P343R in the CH2 region and CH3 region, a variant comprising a modification for substitution of any one or more originally present lysine residues at positions 4, 7, and 35 of the C-domain or Z-domain of Protein A with amino acid residues other than lysine and having an ability to bind to the IgG antibody is a suitable purification tool.

The composition containing the IgG antibody can also be pretreated by filtration, centrifugation or the like before being loaded onto the affinity column. The composition containing the IgG antibody can be loaded onto the affinity column by a general liquid chromatography system at an appropriate pressure and flow rate depending on the size and volume of the column, the size of the carrier, and the like. It is desirable that the amount of the IgG antibody composition loaded onto the affinity column is about the same as the IgG antibody binding capacity of the affinity column. For example, the binding capacity of the IgG antibody can be determined by monitoring the concentration of the IgG antibody flowing through from the affinity column loaded with the composition. More specifically, when the level of the IgG antibody flowing through from the affinity column becomes the same level as the IgG antibody concentration in the loaded composition, it can be determined that the affinity column condition is close to the IgG antibody binding capacity of the affinity column. Efficient purification of the IgG antibody can be expected by then eluting the IgG antibody from the affinity column (step c).

The purification methods of the present invention may additionally comprise the step of washing the affinity column with a washing solution before step (c).

The washing solution is not particularly limited, but the following solution can be used: a combination of a buffer and a salt, which comprises, as a buffer, at least one selected from the group consisting of phosphoric acid, acetic acid, citric acid, glycine, and tris hydroxymethyl aminomethane, and as the salt, at least one selected from the group consisting of arginine, sodium chloride and sodium sulfate.

Purified IgG antibody can be recovered by eluting the IgG antibody adsorbed onto the affinity column (step c), after, as necessary, washing the affinity column. The method of eluting the IgG antibody adsorbed onto the Protein A-modified ligand can be appropriately selected from known conditions. For example, a solution containing at least one selected from the group consisting of hydrochloric acid, acetic acid, citric acid, arginine, glycine or phosphoric acid can be utilized. The concentration of the solution for eluting the IgG antibody from the affinity column can be adjusted as appropriate according to the purpose, and can be, for example, for acetic acid, 20 mM to 500 mM, and usually 50 mM to 200 mM, and for hydrochloric acid, 1 mM to 5 mM. Even while the IgG antibody is eluted from the affinity column, the IgG antibody elution can be traced by monitoring the protein concentration in the eluate.

After step (c), the recovered IgG antibody can be further purified if necessary. For example, the purification methods of the present invention may additionally include a step(s) of purifying the IgG antibody by at least one chromatography selected from the group consisting of cation exchange chromatography, anion exchange chromatography, hydrophobic interaction chromatography, multimode chromatography and hydroxyapatite chromatography.

Through the above steps, in a preferred embodiment, the present invention can isolate an IgG antibody from within or outside (medium, etc.) of host cells and purify it as a substantially pure and homogeneous IgG antibody. More specifically, the present invention provides a method of producing a purified IgG antibody, the method comprising the following steps of:
(i) providing a composition containing an IgG antibody comprising the amino acid residue substitutions Q311R and P343R;
(ii) preparing an affinity column containing a carrier onto which a Protein A-modified ligand is immobilized, wherein the Protein A-modified ligand comprises a modified immunoglobulin-binding domain containing a modification for substitution of any one or more originally present lysine residues at positions 4, 7, and 35 of the C-domain variant of Staphylococcus protein A of SEQ ID NO: 1 or Z-domain of Staphylococcus Protein A of SEQ ID NO: 2 with amino acid residues other than lysine or a multimer of these modified immunoglobulin-binding domains;
(iii) loading the composition containing the IgG antibody onto the affinity column of step (ii); and
(iv) eluting and recovering the IgG antibody from the affinity column loaded with the composition containing the IgG antibody of step (iii).
   The present invention also encompasses highly purified IgG antibodies using the purification methods.

The present invention will be specifically described below with reference to examples, but is not limited thereto.

### EXAMPLE 1

An Antibody A has an increased isoelectric point (pI) and enhanced affinity for Fc receptor IIb (FcRIIb) and neonatal Fc receptors (FcRn) through antibody engineering techniques to improve its pharmacokinetics, which comprises the amino acid residue substitutions of Q311R and P343R in the CH2 and CH3 regions and has the Fc region of SEQ ID NO: 10; however, as a result, it has a weakened binding affinity for Protein A. In other words, although Antibody A has improved its usefulness as a drug by having improved pharmacokinetics, it has new production problems.

The binding affinity of an antibody to Protein A is often used in its purification process (affinity purification). Specifically, the method comprising the step of adsorbing an antibody on a column onto which Protein A has been immobilized, eluting the antibody after washing, and recovering the antibody is widely used as a method for antibody purification. Since Protein A binds to the Fc region of an antibody, it is used for purification of a wide range of antibodies regardless of the antigen specificity of the antibody. Various Protein A columns where the method for immobilizing Protein A, resin, or Protein A itself is modified are commercially available.

In order to find a Protein A-immobilized resin that can be applied to the affinity purification of Antibody A, the affinity of the antibody for the following commercially available Protein A-immobilized resin was compared:
HiTrap MabSelect SuRe (manufactured by GE Healthcare, product name);
ToyoScreen AF-rProtein A HC-650F (manufactured by Tosoh, product name);
Amsphere A3 (manufactured by JSR Life Sciences, registered trademark);
MiniChrom Column Eshmuno A (manufactured by Merck Millipore, registered trademark);
MabSpeed rP202 (manufactured by Mitsubishi Chemical Corporation, registered trademark); and
KanCap Pre-packaged Column (manufactured by Kaneka, product name).
(1) Dynamic Binding Capacity (DBC) of Antibody A in Each Column

Purified Antibody A was dissolved in equilibration buffer and loaded onto a column filled with each Protein A-immobilized resin. The protein concentration of the buffer eluted from the column was traced by ultraviolet light to identify the 5% breakthrough point, and the DBC per 1 L of resin was determined by the following formula. The 5% breakthrough point means the amount of protein loaded onto the column when the protein concentration in the eluate exceeds 5% of the protein concentration in the antibody solution loaded in the column.

$$DBC = \frac{\begin{matrix}Antibody A concentration ( g \text{/} L ) \times \\ loaded liquid volume ( 5 \% breakthrough point ) ( L )\end{matrix}}{Column capacity ( L )}$$

Similarly, for comparison, the DBC of each column was determined for a humanized antibody that did not contain modifications in the Fc region of human IgG1.
(2) Dynamic Binding Capacity; DBC

TABLE 1
DBC of Antibody A in each Protein
A-immobilized Resin (g/L resin)

| Resin | DBC |
|---|---|
| AF-rProtein A HC-650F | 49.1 |
| Amsphere A3 | 13.6 |
| MabSpeed rP202 | 6.4 |
| KanCap A | 2.5 |
| Eshmuno A | 2.3 |
| MabSelect SuRe | 1.9 |
| MabSelect SuRe LX | 1.6 |

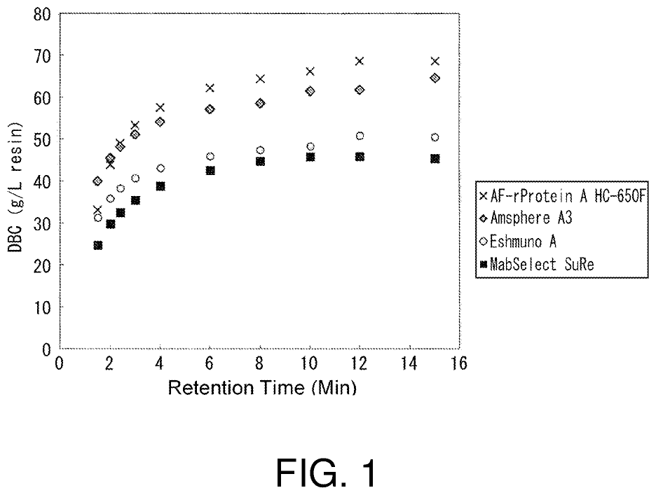

AF-rProtein A HC-650F, which had a large amount of Antibody A bound, showed high DBCs of 46.6 and 45.2 when evaluated also with two resins of different production lots. The next best DBC to AF-rProtein A HC-650F was Amsphere A3 at 13.6. The DBCs of other resins were in the range of 1.6 to 6.4, which are fairly low values. On the other hand, in an antibody in which the Fc region was not modified, the DBCs of each resin were in the range of 20 to 70 as shown in FIG. 1, which were sufficient values for antibody purification.

AF-rProtein A HC-650F (manufactured by Tosoh) is an affinity resin in which an Fc-binding ligand (recombinant of modified Protein A) has been linked to the synthetic polymer carrier TOYOPEARL HW-65. The ligand linked to AF-rProtein A HC-650F has the structure shown in the following Formula (1′):

(R1)₅-(R2)₁ (1′)

In the above Formula (1′), the left end is the N-terminus and the right end is the C-terminus. In the above Formula (1′), (R2) is a modified immunoglobulin-binding domain (SEQ ID NO: 4) in which the amino acid sequence of the C-domain variant (SEQ ID NO: 1), which has been prepared by substituting (G29A) a part of the amino acid sequence of the C-domain of the immunoglobulin-binding domain constituting Protein A derived from Staphylococcus aureus, has been substituted as follows: position 4 with an alanine residue, position 7 with a threonine residue, position 35 with an arginine residue, and position 40 with a lysine residue, position 43 with a lysine residue, position 46 with a lysine residue, and position 53 with a lysine residue. The five (R1) located on the N-terminal side are modified immunoglobulin-binding domains (SEQ ID NO: 5) which, in addition the amino acid sequence of the C-domain variant with substitutions at position 4 with an alanine residue, position 7 with a threonine residue, and position 35 with an arginine residue, has substitutions at position 42 with an alanine residue, position 49 with an arginine residue, position 50 with an arginine residue, and position 58 with an arginine residue. More specifically, Formula (1′) is a ligand consisting of an amino acid sequence in which five of the amino acid sequences of SEQ ID NO: 5 and one amino acid sequence of SEQ ID NO: 4 are linked from the N-terminal side.

In the above-mentioned variant, the binding to the Fc region has been further strengthened by making the modified immunoglobulin-binding domain a hexamer. AF-rProtein A HC-650F has a greatly improved Fc binding property and is an affinity resin with alkali resistance by utilizing the variant of the above structure.

The affinity resins used in the present comparative test are all products where the structure of Protein A itself and the binding mode with its carrier have been optimized, and the Fc region binding property and alkali resistance have been enhanced. It was found that all show excellent binding property to the antibody when an Fc region does not contain any modification, but when an Fc region is modified, there is a big difference in binding property to the antibody depending on the affinity resin.

### EXAMPLE 2

To evaluate the binding performance of ligands to pI-modified antibodies comprising the amino acid residue substitutions Q311R and P343R, the KD value was measured using BLItz (registered trademark) (ForteBio) evaluation system for AF-rProtein A HC-650F and MabSelect SuRe used in Example 1.

The ligand of AF-rProtein A HC-650F (structure shown in Formula (1′)) and the ligand of MabSelect SuRe were each labeled with Biotin (Lys:Biotin =1:1) and then immobilized onto the surface of sensor chips. After equilibration treatment with a PBS solution, the antibody-containing solution was diluted with PBS (+0.1% BSA) buffer and added, and PBS (+BSA) was further added to measure the dissociation reaction.

As the antibody, in addition to Antibody A, tocilizumab (hPM-1 or MRA: see International Patent Application Publication No. WO 92/19759), which is a humanized anti-interleukin 6 (IL-6) receptor antibody, was used as a comparative example. Tocilizumab, unlike Antibody A, does not comprise the amino acid residue substitutions Q311R and P343R for pI modification (a pI-unmodified antibody).

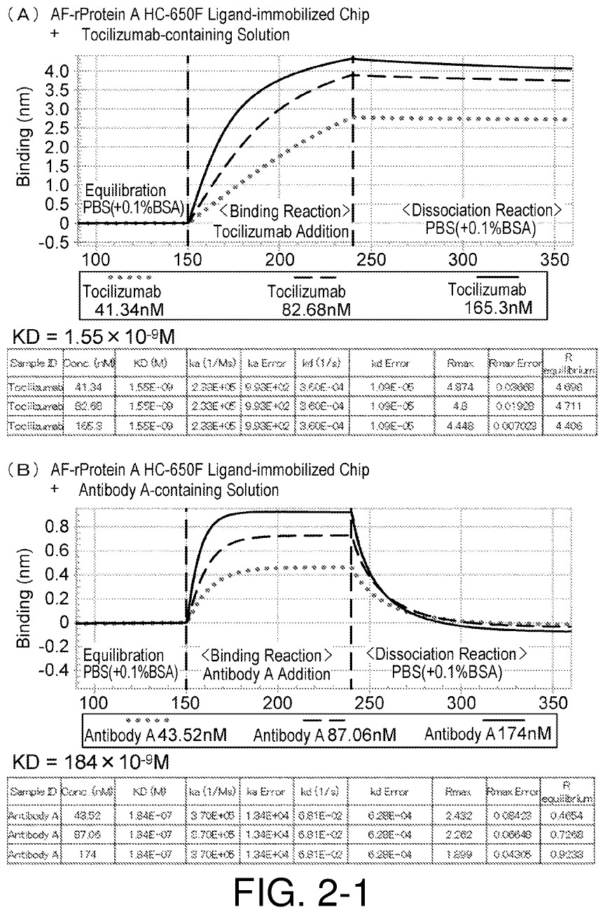

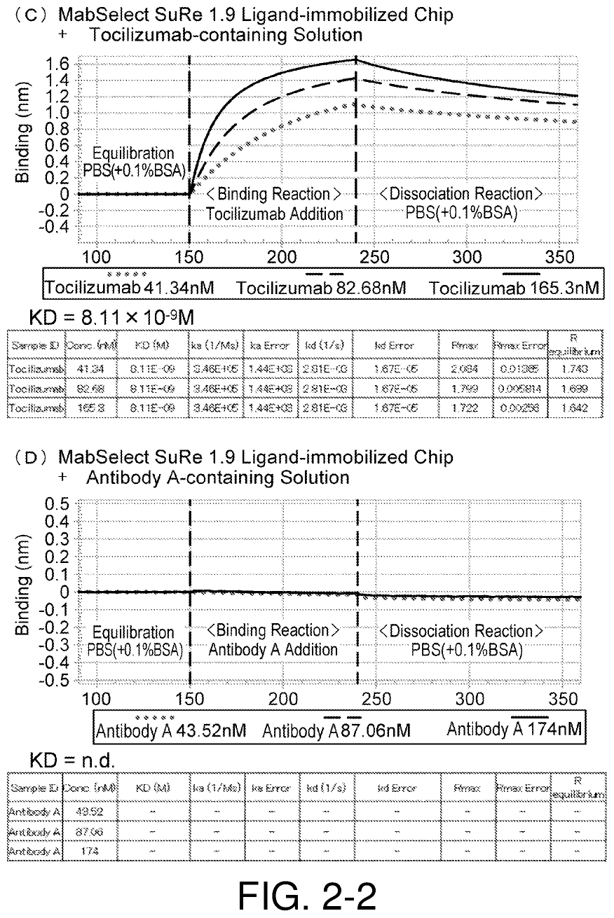

The test results are summarized in FIG. 2 and Table 2. For the AF-rProtein A HC-650F ligand, tocilizumab bound with a K_{D} value of 1.55×10⁻⁹ M. In addition, Antibody A had a K_{D} value of 184×10⁻⁹ M, which although had weaker affinity than tocilizumab, showed binding. On the other hand, regarding the MabSelect SuRe ligand, tocilizumab bound with a KD value of 8.11×10⁻⁹ M, but binding of Antibody A was not observed.

TABLE 2

| | Antibody | |
|---|---|---|
| | Tocilizumab Affinity | Antibody A Affinity |
| Ligand | K_{D} (×10⁻⁹ M) | K_{D} (×10⁻⁹ M) |
| AF-rProtein A HC-650 | 1.55 | 184 |
| MabSelect SuRe | 8.11 | n.d. |

### EXAMPLE 3

In order to evaluate the relationship between the substitution of amino acid residues of the ligand and the binding force to a pI-modified antibody, the KD value of Antibody A was measured by the same method as in Example 2 (however, changing to Lys:Biotin =12:1), for a ligand monomer introduced with mutations at positions 4, 7, and 35 with respect to the C-domain variant (SEQ ID NO: 1).

Results of Table 3 showed that, by substituting the lysine residue (K) at position 35 of the C-domain variant with a glutamine residue (Q) or an arginine residue (R), the affinity for antibody A was increased.

TABLE 3

| | Antibody A | Substitution to C-domain Variant | | |
|---|---|---|---|---|
| | Affinity K_{D} | (SEQ ID NO: 1) | | |
| Ligand | (×10⁻⁴ M) | Position 4 | Position 7 | Position 35 |
| C-domain | n.d. | —* | — | — |
| Variant | | | | |
| PN61 | 1.62 | A | T | R |
| PN34 | 1.99 | — | — | Q |
| PN23 | 1.16 | — | — | R |
| PN43 | n.d. | R | — | — |
| PN44 | n.d. | — | R | — |

*“—” means that there is no substitution to the C-domain variant (position 4: K, position 7: K, position 35: K).

Further, regarding the ligand monomer in which the amino acid residues at positions 40, 43, 46, and 53 of the C-domain variant have been substituted with a lysine residue (K) (hereinafter, “ligand monomer having the R2′ structure”), different mutations were introduced at positions 4, 7, and 35, and the KD value of Antibody A was measured by the same method as in Example 2 (however, changing to Lys:Biotin =12:1).

From the Results of Table 4, among the ligand monomers having the R2′ structure, an increase in affinity for Antibody A was observed by substituting the lysine residue (K) at position 35 with an arginine residue (R) or a valine residue (V), particularly with an arginine residue (R). Furthermore, it was shown that in addition to the substitution at position 35, affinity for Antibody A was increased by substituting the lysine residue (K) at position 7 with any one of a tyrosine residue (Y), a phenylalanine residue (F), a threonine residue (T), an arginine residue (R), a glutamine residue (Q), valine residue (V), leucine residue (L), isoleucine residue (I), histidine residue (H), alanine residue (A), and proline residue (P), particularly with a tyrosine residue (Y) or a phenylalanine residue (F).

TABLE 4

| | Antibody A | Substitution to C-domain Variant (SEQ ID NO: 1) | | | | | | |
|---|---|---|---|---|---|---|---|---|
| | Affinity K_{D} | Position | Position | Position | Position | Position | Position | Position |
| Ligand | (×10⁻⁹ M) | 4 | 7 | 35 | 40 | 43 | 46 | 53 |
| C-domain | n.d. | —* | — | — | — | — | — | — |
| Variant | | | | | | | | |
| R2 | 539 | A | T | R | K | K | K | K |
| PN30 | 42400 | A | — | R | K | K | K | K |
| PN133 | 80100 | A | T | H | K | K | K | K |
| PN190 | 91300 | A | T | A | K | K | K | K |
| PN12 | 39900 | — | — | — | K | K | K | K |
| PN93 | 322 | A | R | R | K | K | K | K |
| PN127 | 265 | A | Q | R | K | K | K | K |
| PN128 | 176000 | A | E | R | K | K | K | K |
| PN132 | 309 | A | V | R | K | K | K | K |
| PN194 | 257000 | A | T | E | K | K | K | K |
| PN195 | 171000 | A | T | T | K | K | K | K |
| PN196 | 1100 | A | T | V | K | K | K | K |
| PN198 | n.d. | A | T | Y | K | K | K | K |
| PN197 | 120000 | A | T | L | K | K | K | K |
| PN129 | 359 | A | L | R | K | K | K | K |
| PN130 | 310 | A | I | R | K | K | K | K |
| PN131 | 314 | A | H | R | K | K | K | K |
| PN2011 | 102 | A | Y | R | K | K | K | K |
| PN2012 | 152 | A | F | R | K | K | K | K |
| PN2078 | 258 | A | A | R | K | K | K | K |
| PN2082 | 1630 | A | P | R | K | K | K | K |

*“—” means that there is no substitution to the C-domain variant (position 4: K, position 7: K, position 35: K, position 40: V, position 43: E, position 46: A, position 53: D).

### EXAMPLE 4

In order to evaluate the relationship between the substitution of amino acid residues of the ligand and the binding force to the pI-modified antibody, the KD value of Antibody A was measured by the same method as in Example 2 (however, changing to Lys:Biotin=12:1) for a plurality of ligand dimers.

The ligand dimers tested had a dimer structure of a ligand monomer having an R2′ structure in which the amino acid residues at positions 40, 43, 46, and 53 have been substituted with a lysine residue (K) with respect to the C-domain variant, and a ligand monomer (hereinafter, “ligand monomer having an R1′ structure”) in which the amino acid residue at position 42 has been substituted with an alanine residue (A) and amino acid residues at positions 49, 50, and 58 have been substituted with an arginine residue (R) with respect to the C-domain variant, and further had mutations at positions 4, 7, and 35. From the results of Table 5, an enhanced affinity for Antibody A was observed for all dimers in which the lysine residue (K) at position 4 was substituted with any one of a valine residue (V), an isoleucine residue (I), a glutamic acid residue (E), and an arginine residue (R).

TABLE 5

| | Antibody A | | Substitution to C-domain Variant (SEQ ID NO: 1) | | | | | | | | | | |
|---|---|---|---|---|---|---|---|---|---|---|---|---|---|
| | Affinity K_{D} | | Position | Position | Position | Position | Position | Position | Position | Position | Position | Position | Position |
| Ligand | (×10⁻⁹ M) | | 4 | 7 | 35 | 40 | 43 | 46 | 53 | 42 | 49 | 50 | 58 |
| PN226 | 306 | R1′ | V | R | R | —* | — | — | — | A | R | R | R |
| | | R2′ | V | R | R | K | K | K | K | — | — | — | — |
| PN229 | 379 | R1′ | I | R | R | — | — | — | — | A | R | R | R |
| | | R2′ | I | R | R | K | K | K | K | — | — | — | — |
| PN231 | 314 | R1′ | E | R | R | — | — | — | — | A | R | R | R |
| | | R2′ | E | R | R | K | K | K | K | — | — | — | — |
| PN232 | 258 | R1′ | R | R | R | — | — | — | — | A | R | R | R |
| | | R2′ | R | R | R | K | K | K | K | — | — | — | — |

*“—” means that there is no substitution for the C-domain variant (position 40: V, position 43: E, position 46: A, position 53: D, position 42: K, position 49: K, position 50: K, position 58: K).

### EXAMPLE 5

In order to evaluate the relationship between the addition of a poly-lysine residue (K) to a ligand monomer and the binding force to a pI-modified antibody, the KD value of Antibody A was measured by the same method as in Example 2 (however, changing to Lys:Biotin=12:1).

The ligand monomer tested had a structure where five lysine residues (K) were added to the C-terminus of a ligand monomer for immobilization onto a carrier, the ligand monomer having an R1′ structure in which the originally present lysine residues at positions 42, 49, 50 and 58 have been substituted with amino acids other than lysine. From the results of Table 6, an enhancement of the affinity for Antibody A was observed by substituting the amino acid at position 35 with an arginine residue in a ligand monomer in which the originally present lysine residues at positions 42, 49, 50 and 58 have been substituted with the non-lysine, alanine residue (A) or arginine residue (R) and a plurality of lysine residues (K) have been added at the C-terminus.

TABLE 6

| | Antibody A | Substitution to C-domain Variant (SEQ ID NO: 1) | | | | | | | |
|---|---|---|---|---|---|---|---|---|---|
| | Affinity K_{D} | Position | Position | Position | Position | Position | Position | Position | Addition to |
| Ligand | (×10⁻⁹ M) | 4 | 7 | 35 | 42 | 49 | 50 | 58 | C-terminus |
| PN100AH | 335 | V | R | R | A | R | R | R | Poly |
| | | | | | | | | | Lys (K) |

### INDUSTRIAL APPLICABILITY

pI-modified antibodies that cannot be purified by Protein A columns commonly-used in industrial production methods can be efficiently and easily purified by using the specific Protein A affinity column based on the present invention. The present invention is useful as a stable and efficient industrial production method of therapeutic antibodies.

1. A method of purifying an IgG antibody comprising the amino acid residue substitutions Q311R and P343R from a composition containing the antibody, wherein the method comprises the steps of:
(a) preparing an affinity column containing a carrier onto which a Protein A-modified ligand is immobilized, wherein the Protein A-modified ligand comprises: a modified immunoglobulin-binding domain comprising a modification for substitution of any one or more originally present lysine residues at positions 4, 7, and 35 of the C-domain variant of Staphylococcus Protein A of SEQ ID NO: 1 or Z-domain of Staphylococcus Protein A of SEQ ID NO: 2 with amino acid residues other than lysine; or a multimer of these modified immunoglobulin-binding domains;
(b) loading the composition containing the IgG antibody onto the affinity column of step (a); and
(c) eluting and recovering the IgG antibody from the affinity column of step (b).

2. The method of claim 1, wherein the multimer of the modified immunoglobulin-binding domains is a dimer to decamer, and wherein arranged at the first or second from the N-terminal or C-terminal side in the multimer is an immunoglobulin-binding domain in which at least one of the originally present amino acid residues at positions 40, 43, 46, 53, 54, and 56 of the C-domain variant (SEQ ID NO: 1) or Z-domain (SEQ ID NO: 2) has been substituted with a lysine residue.

3. The method of claim 1 or 2, wherein the substitution is a modification for substitution of any one or more originally present lysine residues at positions 4, 7, and 35 of the C-domain variant or Z-domain of Protein A with any one of amino acid residues selected from the group consisting of an alanine residue, a glutamine residue, an asparagine residue, a valine residue, a serine residue, a threonine residue, a histidine residue, a tyrosine residue, an arginine residue, a glutamic acid residue, a phenylalanine residue, a leucine residue, an isoleucine residue, and a proline residue.

4. The method of any one of claims 1 to 3, wherein the modified immunoglobulin-binding domain is (i) a modified immunoglobulin-binding domain comprising the amino acid sequence of SEQ ID NO: 3 or 5; or (ii) a modified immunoglobulin-binding domain comprising an amino acid sequence in which one to several amino acid residues have been substituted, deleted, added, and/or inserted to the amino acid sequence of SEQ ID NO: 3 or 5 at amino acid residues other than those at positions 4, 7, and 35.

5. The method of any one of claims 1 to 4, wherein the modified immunoglobulin-binding domain has an ability to bind to an IgG antibody comprising the amino acid residue substitutions Q311R and P343R.

6. The method of any one of claims 1 to 5, wherein the Protein A-modified ligand is a modified ligand comprising a modified immunoglobulin-binding domain that consists of at least one amino acid sequence selected from the group consisting of SEQ ID NOs: 3, 4, and 5.

7. The method of any one of claims 1 to 6, wherein the Protein A-modified ligand is immobilized onto the carrier by any one means selected from the group consisting of (1) to (5) below:
(1) a method of immobilization onto the carrier through a modified immunoglobulin-binding domain in which 1 to 6 of the amino acid residues at positions 40, 43, 46, 53, 54, and 56 in the C-domain variant or Z-domain of Protein A are additionally substituted with a lysine residue;
(2) a method of immobilization onto the carrier through a disulfide bond or a thioether bond by introducing cysteine into the C-terminus of Protein A;
(3) a method of immobilization onto an amino group-containing immobilization carrier by cyanation of a thiol group;
(4) a method of immobilizing a multimer of modified immunoglobulin-binding domains having a cysteine residue onto an amino group-containing carrier using 4-(N-maleimidomethyl)cyclohexane-1-carboxylate (SMCC) as a cross-linking agent; and
(5) a method of immobilization onto the carrier through a plurality of lysine residues added to the C-terminus of a modified immunoglobulin-binding domain in which the lysine residues at positions 42, 49, 50, and 58 of the C-domain variant of Protein A are substituted with amino acids other than lysine, or to a modified immunoglobulin-binding domain in which the lysine residues at positions 49, 50, and 58 of the Z-domain are substituted with amino acids other than lysine.

8. The method of any one of claims 1 to 7, wherein the IgG antibody is an IgG antibody additionally comprising one or more amino acid residue substitutions selected from among M428L, N434A, Y436T, Q438R, and S440E in the CH3 region of the IgG antibody.

9. The method of any one of claims 1 to 8, wherein the IgG antibody is one in which the CH3 region of the IgG antibody comprises an amino acid sequence selected from the group consisting of SEQ ID NOs: 6 to 11.

10. The method of any one of claims 1 to 9, wherein the IgG antibody is one in which the heavy chain constant region of the IgG antibody comprises an amino acid sequence selected from the group consisting of SEQ ID NOs: 12 to 57.

11. The method of any one of claims 1 to 10, wherein the pI value of the antibody is 4.0 to 10.0.


SEQUENCE LISTING (62 sequences)

SEQ ID NO: 1 - protein, 58 aa, Artificial Sequence
  modified C domain of staphylococcal protein A
ADNKFNKEQQNAFYEILHLPNLTEEQRNAFIQSLKDDPSVSKEILAEAKKLNDAQAPK

SEQ ID NO: 2 - protein, 58 aa, Staphylococcus aureus [sequence omitted]

SEQ ID NO: 3 - protein, 58 aa, Artificial Sequence
  modified immunoglobulin-binding domain
ADNAFNTEQQNAFYEILHLPNLTEEQRNAFIQSLRDDPSVSKEILAEAKKLNDAQAPK

SEQ ID NO: 4 - protein, 58 aa, Artificial Sequence
  modified immunoglobulin-binding domain
ADNAFNTEQQNAFYEILHLPNLTEEQRNAFIQSLRDDPSKSKKILKEAKKLNKAQAPK

SEQ ID NO: 5 - protein, 58 aa, Artificial Sequence
  modified immunoglobulin-binding domain
ADNAFNTEQQNAFYEILHLPNLTEEQRNAFIQSLRDDPSVSAEILAEARRLNDAQAPR

SEQ ID NO: 6 - protein, 105 aa, Artificial Sequence
  IgG CH3 region SG1080
GQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 7 - protein, 105 aa, Artificial Sequence
  IgG CH3 region PK55
GQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 8 - protein, 105 aa, Artificial Sequence
  IgG CH3 region PK56
GQRREPQVYTLPPSREEMTKNQVSLTCLVKGFYPSDIAVEWESNGQPENNYKTTPPVLDSDGSFFLYSKLTVRKSRWQQGNVFSCSVMHEALHNHYTQKSLSLSP

SEQ ID NO: 9 - protein, 105 aa, Artificial Sequence
  IgG CH3 region
GQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 10 - protein, 105 aa, Artificial Sequence
  IgG CH3 region TT91
GQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 11 - protein, 105 aa, Artificial Sequence
  IgG CH3 region
GQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 12 - protein, 328 aa, Artificial Sequence
  IgG heavy chain constant region SG1074
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLNGPSVFLFPPKPKDTLMISRTPEVTCVVVDVSDEDPEVKFNWYVDGVEVHNAKTKPREELYNSTYRVVSVLTVLHRDWLNGKEYKCKVSNTALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 13 - protein, 328 aa, Artificial Sequence
  IgG heavy chain constant region SG1077
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELWNGPSVFLFPPKPKDTLMISRTPEVTCVVVDVSDEDPEVKFNWYVDGVEVHNAKTKPREELYNSTYRVVSVLTVLHRDWLNGKEYKCKVSNTALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 14 - protein, 328 aa, Artificial Sequence
  IgG heavy chain constant region SG1080
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLNGPSVFLFPPKPKDTLMISRTPEVTCVVVDVSDEDPEVKFNWYVDGVEVHNAKTKPREELYNSTYRVVSVLTVLHRDWLNGKEYKCKVSNTALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 15 - protein, 328 aa, Artificial Sequence
  IgG heavy chain constant region SG1081
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELWNGPSVFLFPPKPKDTLMISRTPEVTCVVVDVSDEDPEVKFNWYVDGVEVHNAKTKPREELYNSTYRVVSVLTVLHRDWLNGKEYKCKVSNTALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 16 - protein, 328 aa, Artificial Sequence
  IgG heavy chain constant region PK55
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLNGPSVFLFPPKPKDTLMISRTPEVTCVVVDVSDEDPEVKFNWYVDGVEVHNAKTKPREEQYNSTYRVVSVLTVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 17 - protein, 328 aa, Artificial Sequence
  IgG heavy chain constant region PK56
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLNGPSVFLFPPKPKDTLMISRTPEVTCVVVDVSDEDPEVKFNWYVDGVEVHNAKTKPREEQYNSTYRVVSVLTVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVRKSRWQQGNVFSCSVMHEALHNHYTQKSLSLSP

SEQ ID NO: 18 - protein, 328 aa, Artificial Sequence
  IgG heavy chain constant region TT20
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 19 - protein, 328 aa, Artificial Sequence
  IgG heavy chain constant region TT22
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 20 - protein, 328 aa, Artificial Sequence
  IgG heavy chain constant region TT24
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 328 aa, Artificial Sequence
  IgG heavy chain constant region TT26
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 22 - protein, 328 aa, Artificial Sequence
  IgG heavy chain constant region TT28
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 23 - protein, 328 aa, Artificial Sequence
  IgG heavy chain constant region TT30
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 24 - protein, 328 aa, Artificial Sequence
  IgG heavy chain constant region TT32
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 25 - protein, 328 aa, Artificial Sequence
  IgG heavy chain constant region TT34
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 26 - protein, 328 aa, Artificial Sequence
  IgG heavy chain constant region TT66
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 27 - protein, 328 aa, Artificial Sequence
  IgG heavy chain constant region TT70
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 28 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 29 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 30 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 31 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 32 - protein, 328 aa, Artificial Sequence
  IgG heavy chain constant region TT90
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 33 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 34 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 35 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 36 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 37 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 38 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 39 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 40 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 41 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVMHEALHAHTTRKELSLSP

SEQ ID NO: 42 - protein, 328 aa, Artificial Sequence
  IgG heavy chain constant region TT67
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 43 - protein, 328 aa, Artificial Sequence
  IgG heavy chain constant region TT71
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 44 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 45 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 46 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 47 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 48 - protein, 328 aa, Artificial Sequence
  IgG heavy chain constant region TT91
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 49 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 50 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 51 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 52 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 53 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 54 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVVDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 55 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 56 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 57 - protein, 328 aa, Artificial Sequence
  IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVAHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 58 - protein, 330 aa, Homo sapiens [sequence omitted]

SEQ ID NO: 59 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 60 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 61 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 328 aa, Artificial sequence
  An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP